(19) United States
(12) Patent Application Publication
(10) Pub. No.: US 2014/0349317 A1
(43) Pub. Date: Nov. 27, 2014

(54) IMMUNOASSAY METHOD AND IMMUNOASSAY KIT

(71) Applicant: CANON KABUSHIKI KAISHA, Tokyo (JP)

(72) Inventor: Masaaki Kobayashi, Kawasaki-shi (JP)

(73) Assignee: CANON KABUSHIKI KAISHA, Tokyo (JP)

(21) Appl. No.: 14/276,028

(22) Filed: May 13, 2014

(30) Foreign Application Priority Data
May 22, 2013 (JP) 2013-108011

(51) Int. Cl. G01N 33/543; G01N 33/68; G01N 33/558

(52) U.S. Cl. CPC G01N 33/54393; G01N 33/558; G01N 33/6875
USPC 435/7.92; 436/501; 422/69

(57) ABSTRACT

An object of the present invention is to provide an immunoassay method and an immunoassay kit which have the excellent effect of producing enhanced measurement sensitivity and yet reduced non-specific reaction. An immunoassay method including performing immune reaction in the presence of 0.67 to 2.67% (W/V) of chondroitin sulfate and 3.5% (W/V) or more of sodium chloride is provided.

### BACKGROUND OF THE INVENTION

1. Field of the Invention

The present invention relates to an assay method utilizing immune reaction, and relates to an immunoassay method and an immunoassay kit which have the excellent effect of producing enhanced measurement sensitivity and yet reduced non-specific reaction.

2. Description of the Related Art

Immunoassay methods utilizing antigen-antibody reaction are widely used in the field of in vitro diagnostic agents for quantitating trace components in human specimens such as blood.

Many techniques relating to additives have heretofore been disclosed to contrive the enhancement of measurement sensitivity or the suppression of non-specific reaction in immunoassays. For example, Japanese Patent Application Laid-Open No. H02-238361 proposes a method which involves causing chondroitin sulfate to be present in a proportion of 0.1 to 0.3% (W/W) in a sample solution containing a substance to be measured. Japanese Patent No. 3644704 also proposes a method which involves causing sodium chloride to be present in a proportion of 0.94 to 2.4% (W/V) in the sample solution.

### SUMMARY OF THE INVENTION

When the enhancement of measurement sensitivity is contrived in immunoassays, a method involving adding a substance having a reaction-promoting effect to increase a signal value and a method involving adding a substance having a reaction-suppressing effect to decrease background signals are frequently used. However, there has been a paucity of effective methods enabling the enhancement of measurement sensitivity and yet the suppression of non-specific reaction, because the effects of the reaction-promoting agent and the reaction-suppressing agent are in reciprocity with one another. For example, in Japanese Patent Application Laid-Open No. H02-238361, chondroitin sulfate is added for the purpose of enhancing measurement sensitivity, an issue of which is that non-specific reaction is also facilitated as a function of enhancement of the amount added. Hence, a problem has been that chondroitin sulfate cannot be added to an effective concentration at which enhanced measurement sensitivity is observed.

In Japanese Patent No. 3644704, sodium chloride is added for the purpose of suppressing non-specific reaction, an issue of which is that antigen-antibody reaction is also suppressed as a function of enhancement of the amount added. Hence, a problem has been that sodium chloride cannot be added to an effective concentration at which reduced non-specific reaction is observed.

The present invention overcomes the above problems and provides a method and an assay kit which enable the enhancement of measurement sensitivity and yet the suppression of non-specific reaction in immunoassays.

The present invention provides an immunoassay method, including performing immune reaction in the presence of 0.67 to 2.67% (W/V) of chondroitin sulfate and 3.5% (W/V) or more of sodium chloride.

According to the immunoassay method of the present invention, the enhancement of sensitivity and yet the suppression of non-specific reaction are enabled in immunoassays by adding chondroitin sulfate, having a reaction-promoting effect, and sodium chloride, having a reaction-suppressing effect, in proportions of 0.67 to 2.67% (W/V) and 3.5% (W/V) or more, respectively.

According to the immunoassay kit of the present invention, the enhancement of measurement sensitivity and yet the suppression of non-specific reaction are enabled in immunoassays by adding chondroitin sulfate, having a reaction-promoting effect, and sodium chloride, having a reaction-suppressing effect, in proportions of 0.67 to 2.67% (W/V) and 3.5% (W/V) or more, respectively.

Further features of the present invention will become apparent from the following description of exemplary embodiments with reference to the attached drawings.

### BRIEF DESCRIPTION OF THE DRAWINGS

FIG. 1 is a conceptual diagram of an analyzer for the immunoassay kit used in Comparative Examples 1 and 2 and Example 1 of the present invention.

FIG. 2 is a graph summarizing the results of the studies performed in Comparative Examples 1 and 2 and Example 1 of the present invention.

FIG. 3 is a photograph showing the results of the studies performed in Comparative Example 3 of the present invention.

FIG. 4 is a photograph showing the results of the studies performed in Example 2 of the present invention.

### DESCRIPTION OF THE EMBODIMENTS

Preferred embodiments of the present invention will now be described in detail in accordance with the accompanying drawings.

Embodiments of the immunoassay method and the immunoassay kit of the present invention will be described below. The embodiments shown herein are only illustrative and the present invention is not necessarily intended to be limited to these embodiments.

### Embodiment 1

A first embodiment of the present invention is an immunoassay method including performing immune reaction in the presence of 0.67 to 2.67% (W/V) of chondroitin sulfate and 3.5% (W/V) or more of sodium chloride. The sample to be measured in the immunoassay method of the present invention may be any biological sample, such as human and animal blood, serum, plasma, urine, semen, spinal fluid, saliva, sweat, tear, ascites or amniotic fluid, provided that the sample potentially contains a substance to be measured.

The substance to be measured according to the present invention is not particularly limited and may be any substance provided that the substance can be measured by utilizing a well-known antigen-antibody reaction. Examples thereof include nucleic acid, protein, lipid, saccharides, hormone, antibody, receptor and enzyme. More specifically, examples thereof include C-reactive protein (CRP), fibrin degradation products (FDP), cytokines such as interleukin, chemokines, prostate-specific antigen (PSA), high mobility group box 1 (HMGB1), neutrophil elastase, hepatitis B virus and hepatitis C virus.

The immunoassay means the detection or measurement of a substance to be measured by the reaction between the substance to be measured and a substance complementary to the substance, such as the antigen-antibody reaction. The complementary substance means a substance specifically recognizing a substance to be measured and having affinity thereto. For example, if the substance to be measured is an antigen, examples of the complementary substance can include an antibody thereto. In this case, examples of a substance capable of serving as the antigen include protein, peptide, DNA, RNA, and low-molecular compounds. If the substance to be measured is an antibody, examples of the immunologically complementary substance can include an antigen therefor and an antibody thereto. In addition, the combination of a substance to be measured and a substance complementary thereto is not limited provided that they can specifically recognize one another, and examples thereof can include a ligand and a receptor therefor, an antigen and an antibody fragment (or a modified antibody) thereagainst, and a nucleic acid and a nucleic acid complementary thereto.

The immunoassay method of the present invention is not particularly limited, and well-known measurement methods can be utilized. Examples thereof can include enzyme-linked immunosorbent assay (ELISA or EIA), fluorescence immunoassay (FIA), radioimmunoassay (RIA), luminescent immunoassay (LIA), an enzyme antibody technique, a fluorescence antibody technique, an immunochromatographic method, immunonephelometry, latex turbidimetry and a latex agglutination assay. The measurement in the immunoassay method of the present invention may be performed by a hand method or may be carried out using an apparatus such as an analyzer.

For example, it is recommended that the enzyme-linked immunosorbent assay be performed using a microplate on which a first antibody specifically recognizing a substance to be measured is immobilized, a sample diluent, a second antibody specifically recognizing the substance to be measured, modified with an enzyme such as HRP, a wash buffer solution, and a luminescent/chromogenic substrate solution. In the assay, it is also recommended that the enzyme modifying the second antibody be reacted with the substrate under optimal conditions therefor to measure the amount of the enzyme reaction product by an optical method.

It is recommended that the fluorescence immunoassay be performed using an optical waveguide or microplate on which the first antibody specifically recognizing a substance to be measured is immobilized, a sample diluent, the second antibody specifically recognizing the substance to be measured, modified with a fluorescent substance, and a wash buffer solution. In the assay, it is also recommended that the fluorescent substance modifying the second antibody be irradiated with excitation light to measure the intensity of fluorescence emitted by the fluorescent substance. In addition, in the case of the radioimmunoassay, it is recommended to measure the amount of radiation by a radioactive substance by the same operation as in the above-described method, and in the case of the luminescent immunoassay, it is recommended to measure the amount of luminescence by a luminescent reaction system.

In the case of the immunonephelometry, latex turbidimetry, latex agglutination assay or the like, it is recommended to measure transmitted light or scattering light by an end point assay or a rate assay. In the case of the immunochromatographic method, it is recommended to visually confirm the color of a marker appearing on an assay line. An instrument such as an analyzer may be used instead of the visual confirmation.

As used herein, the sample solution means a solution containing or suspected to contain a substance to be measured. The term “immune reaction solution” refers to a solution containing a substance to be measured and a substance complementary thereto. Examples of the sample solution can include solutions potentially containing a substance to be measured, such as human blood, serum, plasma, urine, semen, spinal fluid, saliva, sweat, tear, ascites, amniotic fluid and diluted solutions thereof. In addition, examples of the sample solution include a solution in which the sample is mixed with a buffer solution or the like. The solvent for mixing or dilution may be any of various aqueous solvents. Examples thereof can include purified water, saline and various buffer solutions such as TRIS buffer, phosphate buffer, and phosphate buffered saline. Further, the pH of the buffer solution may be properly selected suitable pH and is not particularly limited; however, the pH ranging from 3 to 12 is commonly selected and used. For the purpose of the structural protection of a substance to be measured, the solvent may properly contain one or more selected from the group consisting of proteins such as bovine serum albumin (BSA), human serum albumin (HAS) and casein, various saccharides, skim milk, various animal sera such as normal rabbit serum, various preservatives such as sodium azide and an antibiotic, and various surfactants such as non-ionic surfactants, amphoteric surfactants and anionic surfactants.

The unit is not limited for performing immune reaction in the presence of 0.67 to 2.67% (W/V) of chondroitin sulfate and 3.5% (W/V) or more of sodium chloride. Chondroitin sulfate and sodium chloride, a substance to be measured and a substance complementary thereto may be added in any order to an immune reaction solution, and chondroitin sulfate, sodium chloride, the substance to be measured and the complementary substance may be added in a concentrated state or a dried state. Specifically, chondroitin sulfate and sodium chloride may be added to a sample solution in advance, followed by adding an antibody; the sample solution and the antibody may be added to a container in which chondroitin sulfate and sodium chloride are present; chondroitin sulfate and sodium chloride may be added to a solution containing the antibody in advance, followed by mixing the resultant with the sample solution; or any other units can be adopted. The effect of chondroitin sulfate and sodium chloride present in a sample solution will now be described.

Chondroitin sulfate has a reaction-promoting effect without regard to the type of the reaction, such as immune reaction or non-specific reaction. Hence, the presence of chondroitin sulfate in the sample solution enables the amplification of a signal value in the immunoassay but also has a disadvantage of amplifying non-specific reaction. In other words, when chondroitin sulfate is present in a high proportion with a view to amplifying the signal of the immune reaction, a signal of the background due to non-specific reaction is also increased, resulting in a reduction in measurement sensitivity.

Sodium chloride has a reaction-suppressing effect without regard to the type of the reaction, such as immune reaction or non-specific reaction. Hence, the presence of sodium chloride in the immune reaction solution enables the decrease of the non-specific reaction but has a disadvantage of also suppressing the immune reaction. In other words, when sodium chloride is present in a high proportion with a view to suppressing the non-specific reaction, a signal value due to the immunoassay is also decreased, resulting in a reduction in measurement sensitivity.

The present invention includes performing immune reaction in the presence of 0.67 to 2.67% (W/V) of chondroitin sulfate and 3.5% (W/V) or more of sodium chloride. As a result of intensive studies, the present inventors have found that sodium chloride, having a reaction-suppressing effect, and chondroitin sulfate, having a reaction-promoting effect, are present in the above-described proportions to provide non-specific reaction-suppressing and yet immune reaction-promoting effects.

The proportion of sodium chloride present in the immune reaction solution can be 3.5% (W/V) or more, which markedly suppresses reaction. The upper limit of the addition proportion is not particularly defined; however, excessive addition should be avoided because sodium chloride also has a salting-out effect. Thus, it can be said that the proportion of sodium chloride can be on the order of 3.5 to 10% (W/V).

The proportion of chondroitin sulfate present in the immune reaction solution can be 0.67 to 2.67% (W/V). The chondroitin sulfate proportion of 0.67% or more can provide a marked reaction-promoting effect. On the other hand, the presence of 3.3% (W/V) or more of chondroitin sulfate extremely increases the viscosity of the sample solution and has a problem with the hand method or the operation of immune reaction using an autoanalyzer. The chondroitin sulfate used in the present invention is chondroitin sulfate or a chloride thereof. It is recommended to use, for example, chondroitin sulfate A, chondroitin sulfate C or chondroitin sulfate C sodium salt.

### Embodiment 2

A second embodiment of the present invention is an immunoassay kit prepared so that immune reaction is performed in the presence of 0.67 to 2.67% (W/V) of chondroitin sulfate and 3.5% (W/V) or more of sodium chloride.

The immunoassay kit of the present invention is not particularly limited provided that the kit is configured so that chondroitin sulfate and sodium chloride are present in proportions of 0.67 to 2.67% (W/V) and 3.5% (W/V) or more, respectively in the immune reaction solution. As described above, chondroitin sulfate and sodium chloride, a substance to be measured and a substance complementary thereto may be added in any order to the immune reaction solution. For example, chondroitin sulfate and sodium chloride may be provided in separate containers and mixed with a sample containing a substance to be measured before measurement for such preparation that the above proportions are achieved, or a method is also possible which involves providing chondroitin sulfate and sodium chloride in the form of a mixture in advance and mixing the mixture with a sample, a diluent, and the like so that the above proportions are achieved. In addition, chondroitin sulfate and sodium chloride may be provided in a dried state and mixed with a sample or a diluent. Further, in the configuration of the immunoassay kit of the present invention, chondroitin sulfate and sodium chloride may be combined with a reagent or may be caused to adhere to a solid phase in advance. For example, in the case of the combination with a reagent, examples of the reagent in the combination include an antibody solution containing a buffer solution, a sample diluent and a marker such as an enzyme, necessary for immune reaction, a reagent containing a substance generating a signal such as coloring, a reagent containing a substance involved in the generation of the signal such as coloring, a reagent containing a substance for calibration, and a reagent containing a substance for accuracy control. Examples of the solid phase can include an insoluble support or test paper used for an immunoassay kit, a microplate, a glass plate, a microtube, a filter paper, and a polymer resin.

The immunoassay kit refers to a kit containing reagents and the like necessary for the immunoassay, and also includes a set of various reagents necessary for the immunoassay, a disposable immunoassay kit packed with various reagents necessary for the immunoassay, a microplate-type immunoassay kit for simultaneously measuring a plurality of samples, and immunochromatography and assay paper containing reagents and the like therein and capable of visually determining results.

As an example of the form of the disposable immunoassay kit, a configuration is possible in which a test container is packed with a spherical or rodlike support on which the first antibody specifically recognizing a substance to be measured is immobilized, solutions of chondroitin sulfate and sodium chloride, a reagent diluent, the second antibody specifically recognizing the substance to be measured, modified with an enzyme such as ALP, a wash solution, a luminescent substrate solution and the like. The shape of the test container is not particularly limited provided that the substance to be measured can be measured. Examples thereof include a scaphoid container in which pluralities of reaction vessels and reagent-housing vessels are arranged, and a channel-type container in which a groove is provided in a plate-like substrate and reaction vessels and housing vessels are connected through channels. The size of the test container is not particularly limited; however, the container can have a small size of on the order of 10 centimeters×10 centimeters or less to use the container by incorporation in the analyzer. In addition, the top of each reaction vessel may also be sealed to avoid the contamination of the reaction vessel with foreign materials and the evaporation/deterioration of the reagent packed in the reagent-housing vessel. For example, there is a method which involves causing an aluminum foil, a polymer film, or the like to adhere to the top of each of the reaction vessel and the housing vessel in the test container. Particularly, sealing can be carried out with an aluminum foil because unsealing can be easily performed at the end of a punching mechanism or a dispensing tip in the analyzer. The material of the test container is not particularly limited provided that the material is not a substance inhibiting reaction for measuring the substance to be measured. Examples thereof include polystyrene resin, polyethylene resin and polypropylene resin.

The following configuration is possible as the form of the microplate-type assay kit. For example, there is a configuration with which a microplate having the first antibody specifically recognizing a substance to be measured immobilized thereon as well as solutions of chondroitin sulfate and sodium chloride, a sample diluent, the second antibody specifically recognizing the substance to be measured, modified with an enzyme such as HRP, a wash solution, a chromogenic substrate solution, and the like are included in the form of reagent bottles. Chondroitin sulfate and sodium chloride may be contained in the sample diluent in advance, or may be caused to adhere in a dried state to the microplate.

The form of immunochromatography can be a lateral flow composed of a housing case, a sample pad, a conjugate pad, a membrane filter, and an absorption pad. The lateral flow is prepared by the following procedure. A first antibody specifically recognizing a substance to be measured labeled with colloidal gold or precolored bead is first prepared, coated on a conjugate pad, and dried. Separately, a second antibody specifically recognizing the substance to be measured is coated on a test line on a membrane filter, and dried. In addition, a third antibody specifically recognizing the second antibody is coated on a control line on the membrane filter, and dried. Finally, the conjugate pad, sample pad and absorption pad are attached to the filter to make a lateral flow.

Then, solutions of chondroitin sulfate and sodium chloride can be included with the lateral flow to make an immunochromatographic assay kit. Solutions of chondroitin sulfate and sodium chloride may be coated on the sample pad or the conjugate pad, dried and attached to the membrane filter to make an immunochromatographic assay kit.

As a more specific aspect of this embodiment, there is, for example, an immunoassay kit containing a chondroitin sulfate solution and a sodium chloride-containing solution and an antibody solution and being such that chondroitin sulfate and sodium chloride have proportions of 0.67 to 2.67% (W/V) and 3.5% (W/V) or more, respectively in the immune reaction. The immunoassay kit can be used in all immunoassays, such as enzyme-linked immunosorbent assay (ELISA or EIA), fluorescence immunoassay (FIA), radioimmunoassay (RIA), luminescent immunoassay (LIA), an enzyme antibody technique, a fluorescence antibody technique, an immunochromatographic method, immunonephelometry, latex turbidimetry and a latex agglutination assay; the antibody can be properly modified according to each method or may be immobilized on a support; and the immunoassay kit may contain latex beads and a microtube, or when used in the immunochromatographic method, the housing case, a sample pad, a conjugate pad, a membrane filter, an absorption pad and colloids, etc.

Alternatively, as another example, there is an immunoassay kit containing a microplate having chondroitin sulfate and sodium chloride attached thereto and an antibody solution and being such that chondroitin sulfate and sodium chloride have proportions of 0.67 to 2.67% (W/V) and 3.5% (W/V) or more, respectively in the immune reaction. The immunoassay kit can be used in immunoassays, such as enzyme-linked immunosorbent assay (ELISA or EIA), fluorescence immunoassay (FIA), radioimmunoassay (RIA) and luminescent immunoassay (LIA); the antibody can be properly modified according to each method; and the immunoassay kit may contain a substrate solution and the like.

### Embodiment 3

A third embodiment of the present invention is a reagent containing chondroitin sulfate and sodium chloride to be added to an immune reaction solution, wherein chondroitin sulfate and sodium chloride have proportions of 0.67 to 2.67% (W/V) and 3.5% (W/V) or more, respectively in the immune reaction. In this embodiment, the form of the reagent is not particularly limited, and may be a solution containing chondroitin sulfate and sodium chloride, or can have a volume and a form suited for a desired assay, such as a tablet, powder, or a reagent attached to a container in a dried state.

### EXAMPLES

The present invention will be specifically described below with reference to Examples. However, the following Examples are only illustrative of the present invention, and the scope of the present invention is not intended to be limited to the following Examples in any manner.

(1) Analyzer for Immunoassay Kit

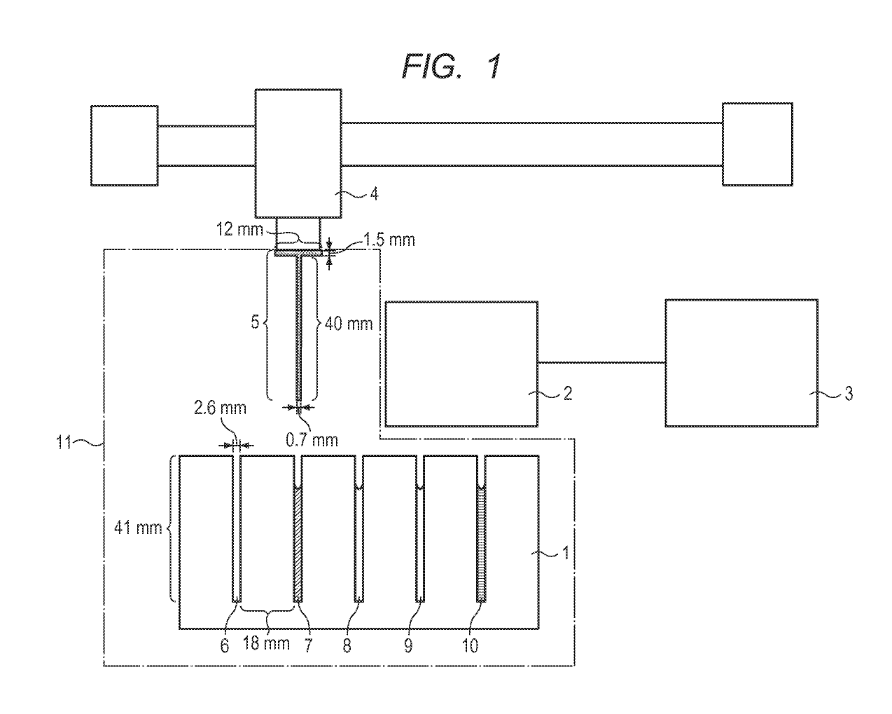

FIG. 1 shows a conceptual diagram of an analyzer for the immunoassay kit prepared in this Example. As shown, a test container 1 is incorporated in the analyzer. In the figure, 2 is a photomultiplier; 3 is a photon counter; and 4 is a handling arm for transferring a columnar insoluble support 5. As shown, the photomultiplier 2 is disposed above the test container 1 to receive light from the luminescent reaction produced in measuring a substance to be measured. The photon counter 3 is placed to measure a signal from the photomultiplier 2 and connected to an external output apparatus. As shown, the handling arm 4 is used in transferring the insoluble support 5 and disposed above the test container 1.

(2) Preparation of Insoluble Support

The insoluble support 5 described in FIG. 1 was prepared by injection-molding a polystyrene resin. The tabular region of the upper part of the support is a tabular region used in transfer by the handling arm 4 and made into a diameter of 12 mm and a thickness of 1.5 mm. The columnar region of the insoluble support 5 is a region for solid-phasing an antibody specifically recognizing a substance to be measured thereon and made in a diameter of 0.7 mm and a length of 40 mm.

(3) Preparation of Test Container

The test container 1 described in FIG. 1 was prepared by injection-molding a polypropylene resin. The test container is provided with a plurality of reaction vessels for performing an immunoassay. Each vessel was made in a diameter of 2.6 mm and a depth of 41 mm. The interval between the contiguous vessels was 18 mm.

(4) Method for Transferring Insoluble Support

The end of the handling arm 4 was equipped with a tubular jig having a silicone rubber-made cylindrical end and a tube (not shown). In addition, the tip of the tube was equipped with a magnetic valve and an air pump. The insoluble support 5 was made liftable/releasable via the tabular region by making the inner part of the tube in negative pressure by the air pump and turning the magnetic valve on and off. The handling arm 4 was moved by the mechanism to enable the insoluble support 5 to move from vessel to vessel in the test container 1.

(5) Immobilization of HMGB1 Antibody on Insoluble Support

HMGB1 polyclonal antibody (Proteintech Group Co., Ltd.) was diluted to 10 μg/ml with Tris buffer (pH 8.0), and 100 μL each was dispensed into a container for immobilizing the antibody. Thereafter, the columnar region of the insoluble support 5 was immersed in the solution and allowed to stand at 4° C. overnight. Then, the insoluble support 5 was taken out and the surface thereof was washed with Tris buffer (pH 8.0). Next, bovine serum albumin (Sigma) was diluted to 5% with Tris buffer (pH 8.0), and 100 μL each was dispensed into a container for immobilizing the antibody. Thereafter, the columnar region of the insoluble support 5 was immersed in the solution and allowed to stand at room temperature for 2 hours. Then, the insoluble support 5 was taken out and the surface thereof was washed with Tris buffer (pH 8.0). Then, sucrose (Wako Pure Chemical Industries Ltd.) was diluted to 5% with Tris buffer (pH 8.0), and 100 μL each was dispensed into a container for immobilizing the antibody. Subsequently, the columnar region of the insoluble support 5 was immersed in the solution and allowed to stand at room temperature for 2 hours. Finally, the insoluble support 5 was taken out, dried and stored at 4° C.

(6) Labeling of ALP on HMGB1 Antibody

Enzyme labeling on HMGB1 monoclonal antibody (Abnova Co., Ltd.) was performed using Alkaline Phosphatase Labeling Kit-NH2 (trade name, Dojindo Laboratories) according to the maker's protocol.

Then, the ALP-labeled HMGB1 monoclonal antibody was adjusted to 2.0 μg/ml using 1% BSA-containing Tris buffer (pH 8.0) and stored at 4° C.

(7) Preparation of Immunoassay Kit

The test container 1 described in FIG. 1 was used in preparing the immunoassay kit 11. The insoluble support 5 on which the HMGB1 polyclonal antibody was immobilized was inserted into the vessel 6 for disposing the insoluble support of the test container. Then, 0.05% Tween 20-containing Tris buffer (pH 8.0) was used as a wash solution to dispense 115 μL thereof into each of washing vessels 8 and 9. Finally, 100 μL of a chemiluminescent reagent (Lumigen) was dispensed into a luminescent reaction vessel 10 to make the immunoassay kit 11.

In using the immunoassay kit 11, an ALP-labeled HMGB1 monoclonal antibody solution adjusted to 2.0 μg/mL (33 μL), a sample containing a substance to be measured (33 μL) and a sample diluent containing chondroitin sulfate and sodium chloride (66 μL) were mixed and dispensed into an immune reaction vessel 7. Thereafter, the resultant was disposed in the analyzer for measurement.

### Reference Example 1

In Reference Example 1, the relation between the proportion (W/V) of sodium chloride in a sample solution and the value of luminescence signal in an immunoassay was examined.

The above-described analyzer was used for measurement. An immunoassay kit was prepared according to (7) above. Human serum solutions containing 0, 3.91, 15.63 and 62.5 ng/mL of HMGB1 were used as samples containing a substance to be measured. Plasma specimens A, B and C from a healthy person were also used. As sample diluents, 3% BSA-Tris buffers (pH 8.0) containing 0, 100, 150, 300, 600, 900, 1,200 and 1,500 mM of sodium chloride were used.

In a measurement method, an ALP-labeled HMGB1 monoclonal antibody solution adjusted to 2.0 μg/mL (33 μL), any of various samples containing a substance to be measured (33 μL) and any of various specimen diluents containing sodium chloride (66 μL) were first mixed and dispensed into the immune reaction vessel 7 in the test container 1. This operation results in the presence of sodium chloride in proportions of 0, 0.39, 0.58, 1.17, 2.34, 3.50, 4.67 and 5.84% (W/V) in the sample solutions. The immunoassay used the one-step sandwich CLEIA method. The reaction was performed under conditions of an immune reaction time of 10 minutes, a washing time of 2 minutes (2 times in total), a luminescent reaction time of 3 minutes and a luminescence measurement time of 6 seconds.

It is understood from Table 1 that the reaction is suppressed without regard to the type of the reaction, such as immune reaction or non-specific reaction, as the proportion of sodium chloride in the sample solution is increased. For example, the value of signal from the plasma specimen from a healthy person is decreased as the proportion of sodium chloride is increased. This means that non-specific reaction is suppressed by sodium chloride. In addition, as shown in the results in measuring 62.5 ng/mL of HMGB1, the value of signal from the human serum specimen is also decreased as the proportion of sodium chloride is increased. This means that immune reaction is also suppressed by sodium chloride. And, the presence of sodium chloride in a proportion of 3.5% (W/V) or more in the sample solution is shown to sufficiently suppress immune reaction and non-specific reaction.

TABLE 1

| Proportion of | Concentration of | Result (Luminescence Intensity) | | | | | |
|---|---|---|---|---|---|---|---|
| Sodium Chloride | Sodium Chloride | Human Plasma Specimen | | | Plasma Specimen from Healthy | | |
| in Sample | in Sample | (HMGB1: ng/mL) | | | Person | | |
| Solution (%) | Diluent (mM) | 3.91 | 15.63 | 62.5 | A | B | C |
| 0 | 0 | 59 | 237 | 2615 | 195 | 885 | 266 |
| 0.39 | 100 | 40 | 181 | 2522 | 187 | 506 | 201 |
| 0.58 | 150 | 16 | 152 | 2487 | 167 | 440 | 201 |
| 1.17 | 300 | 31 | 169 | 1791 | 146 | 316 | 181 |
| 2.34 | 600 | 42 | 198 | 1641 | 136 | 291 | 167 |
| 3.5 | 900 | 42 | 150 | 768 | 169 | 240 | 127 |
| 4.67 | 1200 | 40 | 138 | 792 | 113 | 219 | 126 |
| 5.84 | 1500 | 65 | 287 | 1044 | 108 | 202 | 117 |

### Reference Example 2

In Reference Example 2, the relation between the proportion (W/V) of chondroitin sulfate in a sample solution and the value of luminescence signal in an immunoassay was examined.

The above-described analyzer was used as a measuring apparatus. An immunoassay kit was prepared according to (7) above. In addition, the immunoassay and the preparation of a sample solution were performed as in Reference Example 1.

Human serum solutions containing 0, 3.91, 15.63 and 62.5 ng/mL of HMGB1 were used as samples containing a substance to be measured. 3% BSA-1,500 mM sodium chloride-Tris buffers (pH 8.0) containing 0, 1, 2, 3, 4, 5, 6 and 7% chondroitin sulfate C sodium salt (Wako Pure Chemical Industries Ltd.) were used as sample diluents. This operation results in the presence of chondroitin sulfate in proportions of 0, 0.67, 1.33, 2.00, 2.67, 3.33, 4.00 and 4.67% in the sample solutions.

It is understood from Table 2 that the immune reaction is promoted as the proportion of chondroitin sulfate in the sample solution is increased. For example, as shown in the results in measuring 62.5 ng/mL of HMGB1, the value of signal from the human serum specimen is increased as the proportion of chondroitin sulfate is increased. On the other hand, the proportion of chondroitin sulfate in the sample solution of 3.33% (W/V) or more extremely increased the viscosity of the solution and produced trouble in the operation of washing and the like in the autoanalyzer. Hence, it could be said that the proportion of chondroitin sulfate can be 0.67 to 2.67% (W/V).

TABLE 2

| | | Result | | |
|---|---|---|---|---|
| Proportion of | Proportion of | (Luminescence Intensity) | | |
| Chondroitin Sulfate in | Chondroitin Sulfate in | Human Plasma Specimen | | |
| Sample Solution | Sample Diluent | (HMGB1, ng/mL) | | |
| (%) | (%) | 3.91 | 15.63 | 62.50 |
| 0.00 | 0.00 | −27 | 352 | 1987 |
| 0.67 | 1.00 | 54 | 413 | 2372 |
| 1.33 | 2.00 | 268 | 481 | 3749 |
| 2.00 | 3.00 | 330 | 1164 | 5422 |
| 2.67 | 4.00 | 208 | 2096 | 7985 |
| 3.33 | 5.00 | 1644 | 4363 | 12425 |
| 4.00 | 6.00 | −323 | 2837 | 17350 |
| 4.67 | 7.00 | 1515 | 4700 | 19271 |

### Comparative Example 1

In Comparative Example 1, 3% BSA-Tris buffer (pH 8.0) was used as a sample diluent to measure HMGB1. Because the Tris buffer used contains 0.88% (W/V) of sodium chloride, the preparation of the sample solution described in Reference Examples results in the presence of sodium chloride in a proportion of 0.58%.

The above-described analyzer was used for measurement. An immunoassay kit was prepared according to (7) above. In addition, the immunoassay and the preparation of a sample solution were performed as in Reference Example 1.

Human serum solutions containing 0, 0.24, 0.98, 3.91, 15.63, 62.5, 250 and 1,000 ng/mL of HMGB1 were used as samples containing a substance to be measured.

The results are shown as luminescence intensity in Table 3. When 0.58% sodium chloride was present in the sample solution, the detection limit (±2SD method) was 15.63 ng/mL.

TABLE 3

| | HMGB1 Concentration (ng/ml) | | | | | | | |
|---|---|---|---|---|---|---|---|---|
| | 0.00 | 0.24 | 0.98 | 3.91 | 15.63 | 62.50 | 250.00 | 1000.00 |
| 1 | 95 | 94 | 91 | 103 | 191 | 774 | 5237 | 29141 |
| 2 | 100 | 92 | 89 | 92 | 183 | 689 | 4566 | 30278 |
| 3 | 103 | 96 | 98 | 104 | 172 | 805 | 4749 | 28767 |
| Mean | 99 | 94 | 93 | 100 | 182 | 756 | 4851 | 29395 |
| Standard Deviation | 4 | 2 | 5 | 7 | 10 | 60 | 347 | 787 |
| CV % | 4.07 | 2.13 | 5.10 | 6.68 | 5.24 | 7.94 | 7.15 | 2.68 |
| ΔBlank | 0 | −5 | −7 | 0 | 83 | 657 | 4751 | 29296 |
| Detection Limit | | −17.4 | −24.2 | −21.1 | 55.5 | 528.5 | 4049.5 | 27714.0 |
| (±2SD Method) | | | | | | | | |
| HMGB1 Concentration Ratio | 0.00000 | 0.00024 | 0.00098 | 0.00391 | 0.01563 | 0.06250 | 0.25000 | 1.00000 |
| Luminescence Signal Ratio | | | | | 0.00282 | 0.02241 | 0.16218 | 1.00000 |

### Comparative Example 2

In Comparative Example 2, 3% BSA-Tris buffer (pH 8.0) containing 0.2% chondroitin sulfate was used as a sample diluent to measure HMGB1. Because the Tris buffer used contains 0.88% of sodium chloride, the preparation of the sample solution described in Reference Examples results in the presence of chondroitin sulfate and sodium chloride in proportions of 0.13% and 0.58%, respectively.

The above-described analyzer was used for measurement. An immunoassay kit was prepared according to (7) above. In addition, the immunoassay and the preparation of a sample solution were performed as in Reference Example 1. The same samples as in Comparative Example 1 were used as samples containing the substance to be measured.

The results are shown as luminescence intensity in Table 4. When 0.13% chondroitin sulfate and 0.58% sodium chloride were contained in the sample solution, the detection limit (±2SD method) was 15.63 ng/mL.

TABLE 4

| | HMGB1 Concentration (ng/ml) | | | | | | | |
|---|---|---|---|---|---|---|---|---|
| | 0.00 | 0.24 | 0.98 | 3.91 | 15.63 | 62.50 | 250.00 | 1000.00 |
| 1 | 109 | 110 | 114 | 124 | 224 | 1142 | 5182 | 18249 |
| 2 | 116 | 115 | 105 | 116 | 191 | 1002 | 5264 | 18954 |
| 3 | 113 | 115 | 117 | 119 | 215 | 977 | 4799 | 17480 |
| Mean | 113 | 113 | 112 | 120 | 210 | 1040 | 5082 | 18228 |
| Standard Deviation | 4 | 3 | 6 | 4 | 17 | 89 | 248 | 737 |
| CV % | 3.12 | 2.55 | 5.58 | 3.38 | 8.12 | 8.55 | 4.88 | 4.04 |
| ΔBlank | 0 | 1 | −1 | 7 | 97 | 928 | 4969 | 18115 |
| Detection Limit | | −12.1 | −20.2 | −8.1 | 56.2 | 742.8 | 4465.6 | 16633.5 |
| (±2SD Method) | | | | | | | | |
| HMGB1 Concentration Ratio | 0.00000 | 0.00024 | 0.00098 | 0.00391 | 0.01563 | 0.06250 | 0.25000 | 1.00000 |
| Luminescence Signal Ratio | | | | | 0.00537 | 0.05121 | 0.27430 | 1.00000 |

### Example 1

In Example 1, HMGB1 was measured using 3% BSA-Tris buffer (pH 8.0) containing 3% chondroitin sulfate and 1,500 mM sodium chloride as a sample diluent.

The above-described analyzer was used as a measuring apparatus. An immunoassay kit was prepared according to (7) above. In addition, the immunoassay and the preparation of a sample solution were performed as in Reference Example 1. The same samples as in Comparative Example 1 were used as samples containing the substance to be measured.

The results are shown in Table 5. When 2% chondroitin sulfate and 5.8% sodium chloride were contained in the sample solution, the detection limit (±2SD method) was 3.91 ng/mL.

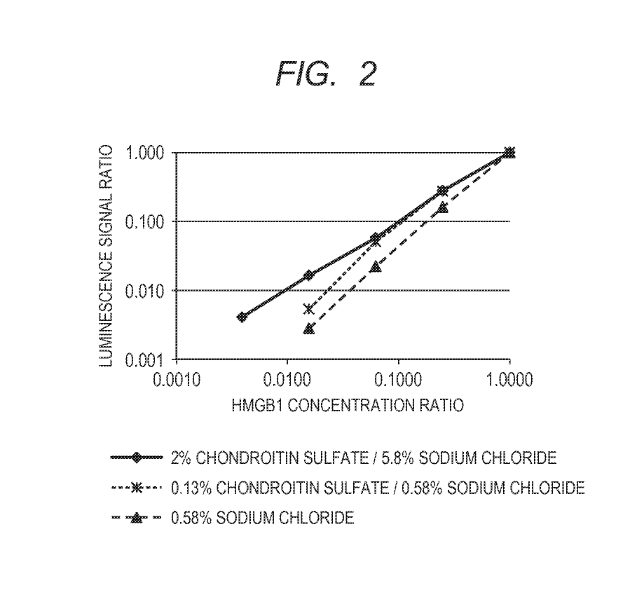

FIG. 2 shows the measurement results obtained in Comparative Examples 1 and 2 and Example 1. To facilitate the comparison of the results obtained, the X axis of the graph was expressed as an HMGB1 concentration ratio (concentration ratio when the HMGB1 concentration of 1,000 ng/mL was assumed as 1) and the Y axis, as a luminescence signal ratio (signal ratio when the signal value obtained in measuring the HMGB1 concentration of 1,000 ng/mL was assumed as 1).

A higher signal can be obtained when 0.13% chondroitin sulfate was contained in the sample solution than when only 0.58% sodium chloride was contained therein. However, this concentration of chondroitin sulfate is not effective to the extent of enhancing the detection limit. On the other hand, it turns out that the detection limit is enhanced when 2% chondroitin sulfate and 5.8% sodium chloride are contained in the sample solution.

As described above, the immunoassay kit of the present invention can be used to enhance measurement sensitivity and yet suppress non-specific reaction.

TABLE 5

| | HMGB1 Concentration (ng/ml) | | | | | | | |
|---|---|---|---|---|---|---|---|---|
| | 0.00 | 0.24 | 0.98 | 3.91 | 15.63 | 62.50 | 250.00 | 1000.00 |
| 1 | 151 | 149 | 160 | 173 | 209 | 365 | 1374 | 4430 |
| 2 | 150 | 148 | 151 | 169 | 210 | 420 | 1312 | 4317 |
| 3 | 146 | 145 | 161 | 162 | 237 | 393 | 1289 | 4372 |
| Mean | 149 | 147 | 157 | 168 | 219 | 393 | 1325 | 4373 |
| Standard Deviation | 3 | 2 | 6 | 6 | 16 | 28 | 44 | 57 |
| CV % | 1.78 | 1.41 | 3.50 | 3.31 | 7.26 | 7.00 | 3.32 | 1.29 |
| ΔBlank | 0 | −2 | 8 | 19 | 70 | 244 | 1176 | 4224 |
| Detection Limit | | −11.1 | −8.0 | 2.6 | 32.6 | 183.4 | 1082.8 | 4105.7 |
| (±2SD Method) | | | | | | | | |
| HMGB1 Concentration Ratio | 0.00000 | 0.00024 | 0.00098 | 0.00391 | 0.01563 | 0.06250 | 0.25000 | 1.00000 |
| Luminescence Signal Ratio | | | | 0.00450 | 0.01649 | 0.05769 | 0.27841 | 1.00000 |

(8) Preparation of HMGB1 Monoclonal Antibody-Sensitized Colloid

9 mL of a colloidal gold solution (BBI Ltd., particle diameter: 40 nm) and 1 mL of 20 mM phosphate buffer (pH 7.0) were added to a 50 mL-tube for centrifugation and slightly stirred. Next, 1 mL of 80 μg/mL HMGB1 monoclonal antibody was added thereto, which was then allowed to stand at room temperature for 10 minutes. Thereafter, 0.55 mL of 1% PEG20000 (Kishida Chemical Co., Ltd.) and 1.1 mL of 10% BSA were added thereto, which was then slightly stirred. The solution was centrifuged under conditions of 8,000 g—15 minutes to precipitate the colloidal gold, followed by removing the supernatant. 20 mL of pH 8.2 colloidal gold storage buffer (an aqueous solution of 5 g of BSA, 0.5 g of sodium azide, 0.25 g of PEG20000, 1.211 g of tris(hydroxymethyl)aminomethane and 4.383 g of sodium chloride dissolved in 500 mL of pure water) was added thereto, which was again centrifuged. Thereafter, the supernatant was removed, and the resultant colloidal gold-sensitized HMGB1 monoclonal antibody was diluted to an absorbance (520 nm) of 6.0 with the colloidal gold storage buffer.

(9) Preparation of Half Strip on Which HMGB1 Polyclonal Antibody Is Immobilized

An absorption pad (Nihon Millipore K.K., 17 mm×30 cm) was affixed to the absorption pad adhesion site of a membrane card (Nihon Millipore K.K., High-Flow Plus membrane card, 6 cm×30 cm). The membrane card was then subjected to cutting off below the conjugate pad adhesion site thereof. In addition, the membrane card was cut to a width of about 5 mm and used as a half strip immunochromatogram.

The coating of antibody on places to be used as test and control lines was carried out by dropwise addition using a micropipettor. First, 0.5 μL of 0.5 mg/mL HMGB1 polyclonal antibody was added dropwise to a position about 10 mm from each end (the above cut site) and the resultant was used for the test line. Then, 0.5 μL of 0.5 mg/mL anti-mouse IgG antibody (LifeSpan Bioscience Ltd.) was added dropwise about 5 mm apart from the test line and the resultant was used for a control line.

### Comparative Example 3

In Comparative Example 3, the chromatography measurement of HMGB1 was carried out using 3% BSA-Tris buffer (pH 8.0) containing 2% chondroitin sulfate as a sample diluent.

Human serum solutions containing 0, 0.24, 0.98, 3.91, 15.63, 62.5, 250 and 1,000 ng/mL of HMGB1 were used as samples containing a substance to be measured.

The measurement method is as follows. First, an HMGB1 monoclonal antibody-sensitized colloid solution (10 μL), the above sample diluent (50 μL) and any of various samples containing the substance to be measured (50 μL) were mixed and dispensed into a 96-well microplate. Then, the prepared half strip on which HMGB1 polyclonal antibody was immobilized was immersed therein to develop the mixture on the membrane. Then, 40 minutes after immersion, the half strip was taken out and the presence of the test line was visually confirmed. The sample diluent and HMGB1 monoclonal antibody-sensitized colloid solution used contain 0.88% sodium chloride. Thus, chondroitin sulfate and sodium chloride will be present in proportions of 0.9% and 0.48%, respectively in the sample solution.

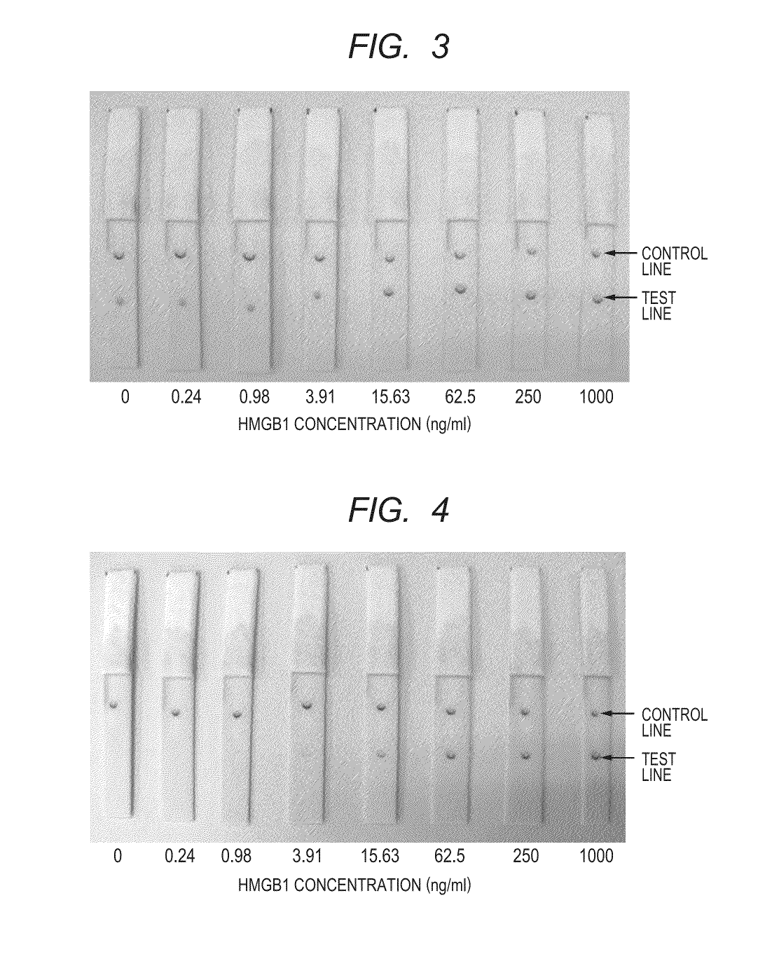

The measurement results are shown in FIG. 3. When HMGB1 at a concentration of 0 ng/mL was measured, pseudopositive reaction occurred in the test line, and thus qualitative determination could not be performed under these conditions. This shows that non-specific reaction is not suppressed and pseudopositive reaction occurs when sodium chloride is not present in a suitable proportion even in the presence of chondroitin sulfate in a suitable proportion in each sample solution.

### Example 2

In Example 2, the immnochromatography measurement of HMGB1 was carried out using 3% BSA-Tris buffer (pH 8.0) containing 2% chondroitin sulfate and 1,500 mM sodium chloride as a sample diluent.

The same samples as in Comparative Example 3 were used as samples containing the substance to be measured. The measurement method was also carried out as in Comparative Example 3. The HMGB1 monoclonal antibody-sensitized colloid solution used contains 0.88% sodium chloride. Thus, chondroitin sulfate and sodium chloride will be present in proportions of 0.9% and 4.1%, respectively in the sample solution.

The measurement results are shown in FIG. 4. When HMGB1 at a concentration of 0 ng/mL was measured, no pseudopositive reaction (non-specific reaction) was observed in the test line. It was possible to visually determine HMGB1 from a concentration of 3.91 ng/ml.

As described above, the immunoassay kit of the present invention can be used to enhance measurement sensitivity and yet suppress non-specific reaction.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-108011, filed May 22, 2013, which is hereby incorporated by reference herein in its entirety.

What is claimed is:

1. An immunoassay method for a substance to be measured, comprising performing immune reaction in the presence of 0.67 to 2.67% (W/V) of chondroitin sulfate and 3.5% (W/V) or more of sodium chloride.

2. The immunoassay method according to claim 1, wherein the substance to be measured is HMGB1.

3. An immunoassay kit prepared so that immune reaction is performed in the presence of 0.67 to 2.67% (W/V) of chondroitin sulfate and 3.5% (W/V) or more of sodium chloride.

4. The immunoassay kit according to claim 3, at least including a sample diluent containing chondroitin sulfate and sodium chloride, an insoluble support on which an antibody specifically recognizing a substance to be measured is immobilized, and a test container, wherein:
in diluting a sample solution with the sample diluent,
a sample-sample diluent mixture is prepared so that chondroitin sulfate and sodium chloride have proportions of 0.67 to 2.67% (W/V) and 3.5% (W/V) or more, respectively.

5. The immunoassay kit according to claim 4, wherein the substance to be measured is HMGB1.

6. The immunoassay kit according to claim 3, wherein:
the immunoassay kit is an immunochromatographic assay kit; and
the immunoassay kit includes at least a sample diluent containing chondroitin sulfate and sodium chloride and a lateral flow on which an antibody specifically recognizing a substance to be measured is immobilized, wherein:
in diluting a sample solution with the sample diluent,
the sample-sample diluent mixture is prepared so that chondroitin sulfate and sodium chloride have proportions of 0.67 to 2.67% (W/V) and 3.5% (W/V) or more, respectively.

7. The immunoassay kit according to claim 6, wherein the substance to be measured is HMGB1.
